(19) United States
(12) Patent Application Publication
(10) Pub. No.: US 2020/0038618 A1
(43) Pub. Date: Feb. 6, 2020

(54) Breathing Assistance Device with Nebulizer

(71) Applicant: Mercury Enterprises, Inc., Clearwater, FL (US)

(72) Inventor: Jeffrey Bruce Ratner, Pinellas Park, FL (US)

(21) Appl. No.: 16/594,689

(22) Filed: Oct. 7, 2019

Related U.S. Application Data
(63) Continuation-in-part of application No. 15/340,142, filed on Nov. 1, 2016, now abandoned.
Application No. 15/340,142 is a continuation of application No. 13/693,638, filed on Dec. 4, 2012, now Pat. No. 9,511,202.

(51) Int. Cl. A61M 16/14; A61M 11/06; A61M 16/20; A61M 11/02; A61M 16/00; A61M 16/06; A61M 16/08

(52) U.S. Cl. CPC A61M 16/14; A61M 11/06; A61M 16/209; A61M 11/02; A61M 2016/0027; A61M 16/06; A61M 16/0875; A61M 16/20; A61M 16/0057

(57) ABSTRACT

A breathing assistance device includes a patient breathing tube in fluid communication with a face mask. A source of air connected to a first port formed integrally with a valve in fluid communication with the breathing tube and a nebulizer is connected to a second port formed integrally with the valve. A reservoir holding medicine in liquid form is connected to the nebulizer. A valve actuator has an open configuration where gaseous fluid and aerosolized medicine from the nebulizer flow to the patient through the breathing tube and a closed configuration where only gaseous fluid is delivered. Medication can, therefore, be selectively administered to the patient by manipulating the valve actuator. The valve enables the patient to exhale against pressure slightly above atmospheric pressure. Filter device is secured to the valve body and is configured to filter contaminates from a patients exhaled breath prior to being exhausted into the environment.

### CROSS-REFERENCE TO RELATED APPLICATIONS

This nonprovisional application is a continuation-in-part of and claims priority to nonprovisional application Ser. No. 15/340,142, entitled “BREATHING ASSISTANCE DEVICE WITH NEBULIZER,” filed Nov. 1, 2016 by the same inventor, which is a continuation of nonprovisional application Ser. No. 13/693,638, entitled “BREATHING ASSISTANCE DEVICE WITH NEBULIZER,” filed Dec. 4, 2012, issued on Dec. 6, 2016 as U.S. Pat. No. 9,511,202 by the same inventor.

### BACKGROUND OF THE INVENTION

### 1. Field of the Invention

This invention relates, generally, to devices that help patients breathe. More particularly, it relates to a breathing assistance device that includes a nebulizer and a filter, but which does not require a second flow valve connection.

### 2. Description of the Prior Art

A conventional breathing assistance device delivers preselected gaseous fluids suitable for respiration to a patient who requires breathing assistance. The preselected gaseous fluids are stored under pressure in a reservoir, which may hold oxygen or miscellaneous mixtures of suitable gaseous fluids. The patient wears a facemask and a tube interconnects the reservoir and the facemask. A manometer and a pressure relief valve may be provided between the tank and the facemask and such parts perform their respective well-known functions.

At times a physician may want to employ a nebulizer to administer therapeutic dosages of a medicine to a patient suffering from breathing difficulties. However, there are a number of problems associated with the prior art use of nebulizers. First, the flow of gaseous fluid to the patient must be interrupted so that a nebulizer can be added to the system. Secondly, the nebulizer must be connected to a second reservoir of gaseous fluid if the reservoir already in use does not have a “Y” connection so that the nebulizer flow valve can be attached thereto. Third, prior nebulizers, including the previous related application (U.S. application Ser. No. 15/340,142), allow for the patients potentially harmful exhaled gaseous fluid (e.g., exhaled breath) and/or aerosolized medication to be freely discharged into the environment surrounding the patient, including medical personnel. This discharge of potentially contaminated air increases the risks that third parties, such as medical personnel may contract a bacterial and/or viral infection from the patient if the patient has been admitted for a bacterial and/or viral infection. Fourth, it is undesirable, harmful, and possibly fatal for aerosolized medication to be inhaled by an individual, such as hospital staff, whom the medication is not prescribed. Adverse reactions, such as anaphylactic shock, resulting from an allergic reaction to the medication may occur.

Thus, there is a need for a breathing assistance device that enables a patient to receive medical treatment from a nebulizer without interrupting the flow of gaseous fluid to the patient and simultaneously prevents the patient's unfiltered exhaled breath from contaminating an environment occupied by the patient and medical personnel.

There is also a need for a system that does not require connection to a second flow valve associated with a second reservoir as described in an embodiment of the invention. Moreover, there is a need for a system that does not require that a reservoir in use be equipped with a “Y” connection thereby allowing a second flow valve to be connected to the reservoir as described in an embodiment of the invention.

In view of the art considered as a whole at the time the present invention was made, it was not obvious to those of ordinary skill how medical treatment could be administered to a patient wearing a breathing assistance device without requiring a second reservoir or a reservoir having a “Y” connection.

### SUMMARY OF THE INVENTION

The long-standing but heretofore unfulfilled need for a breathing assistance device that is used with a single tank having a single flow control valve, which does not require interruption of flow of gaseous fluid to a patient and filters a patient's exhalation breath prior to discharging the expiration gas into the environment is now met by a new, useful, and nonobvious invention.

The novel breathing assistance device includes a patient breathing tube having a leading end adapted to be in fluid communication with a facemask worn by a patient who requires breathing assistance. A nebulizer port is connected to or formed integrally with the patient breathing tube. In an embodiment, the nebulizer port extends radially away from the patient's breathing tube. The nebulizer has a leading end formed integrally with or releasably attached to the nebulizer port and includes a reservoir adapted to hold a liquid medical compound.

A valve body is coupled to the trailing end of the patient's breathing tube and maybe in axial alignment therewith. A first port is in fluid communication with the valve body. In an embodiment, the first port extends radially relative to a longitudinal axis of the valve body, however, one of ordinary skill in the art may appreciate that the first port may be oriented away from the valve body in any direction.

A first tube has a trailing end connected in fluid communication with a remote source of gaseous fluid under pressure and includes a leading end connected in fluid communication to the first port. A second port is in fluid communication with the valve body and extends radially relative to the longitudinal axis of the valve body in a circumferentially spaced relation to the first port, however, any other orientation of the second port relative to the valve body and the first port is contemplated.

A second tube has a trailing end in fluid communication with the second port and a leading end in fluid communication with a trailing end of the nebulizer, i.e., to the fluid reservoir of the nebulizer.

A valve may be positioned between the reservoir and the facemask and includes both an open and a closed configuration. In the closed configuration, the valve prevents the flow of gaseous fluid through the second tube and in the open configuration, the valve enables the flow of gaseous fluid through the second tube. The position of the valve does not alter the flow of gaseous fluid from the reservoir to the facemask through the first tube.

The valve may be a gate valve, a butterfly valve, or any other type of valve that is suitable for controlling a flow of a fluid. The valve may be selectively opened and closed by a valve actuator. The valve actuator, for example, maybe a hand-operated rotatable valve actuator, a hand-operated slide valve actuator, a hand-operated toggle valve actuator, and the like. In an embodiment, electrical or electronic means may operate the valve actuator, however, it is preferable to have a hand-operated valve actuator to limit the complexity of the system and provide a simple and intuitive means for transitioning the valve from the open to the closed configuration and vice versa.

In an embodiment, the valve includes a handle that is rotatably mounted relative to a longitudinal axis of the patient's breathing tube and the valve body. In such an embodiment, a ninety-degree (90°) rotation of the handle in a first direction opens the valve and allows for gaseous fluid to flow through the second tube. Consequently, a ninety-degree (90°) rotation in the opposite direction closes the valve and prevents the flow of gaseous fluid through the second tube.

Accordingly, the novel breathing assistance device delivers a gaseous fluid under pressure from the remote reservoir to a patient when the valve is in the closed configuration, similarly to how the system would operate if no nebulizer was used.

The novel breathing assistance device in the open configuration delivers gaseous fluid under pressure from the remote reservoir to the patient along with the aerosolized medication from the nebulizer. The spray of the aerosolized medicine from the nebulizer is entrained into the flow of air flowing through the patient's breathing tube to the patient's face mask.

In an embodiment, the valve includes a shaft formed integrally with the valve and is axially displaced when the valve is rotated. One or more O-rings may be mounted on the shaft in longitudinally spaced relation to one another to provide a sealing function when the valve is in the closed configuration thereby preventing the flow of gaseous fluid through the second tube. One or more O-rings provide no sealing function when the valve is open configuration, thereby allowing the flow of the gaseous fluid under pressure through the second tube to the nebulizer. In an embodiment, O-rings may be disposed at least partially within an outer surface of the shaft.

In an embodiment, a plurality of ramps are formed integrally with the trailing end of the valve. The handle has a disc-shaped leading end and a plurality of pins disposed circumferentially and formed integrally with the leading end. The pins extend away from a longitudinal axis of the device.

The valve cover has a plurality of ramps configured to mate with the trailing end of the valve. The plurality of ramps in the valve cover matches the size, quantity, and slope of the ramps formed in the trailing end of the valve.

The pins are sandwiched between the ramps formed in the trailing end of the valve and the ramps formed in the leading end of the valve cover. The pins translate in a sliding motion up their associated ramps when the valve handle is rotated from a first position to a second position. The pins translate down their associated ramps in a sliding motion when the valve handle is rotated from the second configuration to the first configuration. This constrains the shaft to only be displaced axially along its longitudinal axis when the valve handle is rotated. Any electrical, electronic, hydraulic, or mechanical means for translating rotational movement of a valve handle to axial translation of a shaft for the purpose of opening and closing the valve is contemplated herein.

Moreover, any electrical, electronic, hydraulic, or mechanical means for opening and closing a valve that controls the path of travel of gaseous fluid is within the scope of this invention. The invention provides a caregiver with the ability to switch between a main gaseous flow from a single remote reservoir having a single flow valve and no “Y” connection to a patient where a nebulizer is bypassed to gaseous flow from the same single remote reservoir to the patent where at least some of the gaseous flow is routed through a nebulizer without interrupting the main gaseous flow.

An orifice jet is disposed concentrically within a lumen of the patient's breathing tube. The orifice jet is in fluid communication with the remote source of gaseous fluid under pressure when the valve is in the open configuration.

A plurality of slots, rectangular openings, or an outlet port is formed in a leading end of the valve body, the openings enable the patient to exhale into the environment surrounding the patient when the nebulizer valve is in either the open or the closed configurations. In an embodiment, a filter device is connected to the outlet port thereby filtering the patient's exhalation breath prior to exhausting the patient's breath into the environment. In an embodiment, the filter device includes a housing, a cover, and at least one filter residing between the housing and the cover, such that prior to being exhausted through the cover, the gaseous liquid is disposed through the at least one filter.

The patient receives a combination of gaseous fluids from the orifice jet and entrained ambient air through the openings when the patient inhales at a rate greater than the rate that gaseous fluid is delivered from the remote source of gaseous fluid under pressure and no gaseous fluid is flowing through the nebulizer tube.

The patient receives a combination of gaseous fluid from the orifice jet, entrained room air through the openings formed in the leading end of the valve, and from the nebulizer, together with aerosolized medication if the gaseous fluid is flowing through the nebulizer.

In an embodiment of the invention, the device enables a patient wearing a facemask receiving assistance in breathing to be administered one or more medications as needed without interruption of the flow of the gaseous fluids to the patient.

In an embodiment of the invention, the device is a breathing assistance device that is equipped with a built-in nebulizer having a manually operated valve for switching between an open configuration and a closed configuration.

In an embodiment of the invention, a breathing assistance device is equipped with a built-in nebulizer operating from a single remote reservoir having a single flow valve.

In an embodiment of the invention, a breathing assistance device delivers aerosolized medication to a patient when the nebulizer valve is in the open configuration and restricts the flow of aerosolized medication when the nebulizer valve is in the closed configuration.

In an embodiment of the invention, a breathing assistance device enables a patient to exhale against a pressure greater than atmospheric pressure.

These and other important objects, advantages, and features of the invention will become clear as this disclosure proceeds.

The invention accordingly comprises the features of construction, the combination of elements, and arrangement of parts that will be exemplified in the disclosure set forth hereinafter and the scope of the invention will be indicated in the claims.

### BRIEF DESCRIPTION OF THE DRAWINGS

For a fuller understanding of the nature and objects of the invention, reference should be made to the following detailed disclosure, taken in connection with the accompanying drawings, in which:

FIG. 1 is a front perspective view of the device.

FIG. 2A is a first rear perspective view of the device, depicting a flow control valve in its open position.

FIG. 2B is a second rear perspective view of the device, depicting the flow control valve in its closed position

FIG. 3 is a longitudinal sectional view taken along line 3-3 in FIG. 1.

FIG. 4A is an exploded front perspective view of the novel components.

FIG. 4B is an exploded rear perspective view of the novel components.

FIG. 4C is an exploded view of an embodiment of the components, depicting rectangular openings formed within valve body.

FIG. 5A is a sectional view taken along line 5A-5A in FIG. 4A.

FIG. 5B is a sectional view taken along line 5B-5B in FIG. 4B.

FIG. 6A is a perspective view of the novel components when the flow control valve is in its closed configuration.

FIG. 6B is a perspective view of the novel components when the flow control valve is in its open configuration.

FIG. 7 is a perspective view of the valve cover;

FIG. 8 is an exploded perspective view of the component of the filter and the novel components.

FIG. 9 is a front perspective exploded view of the filter components.

### DETAILED DESCRIPTION OF THE PREFERRED EMBODIMENT

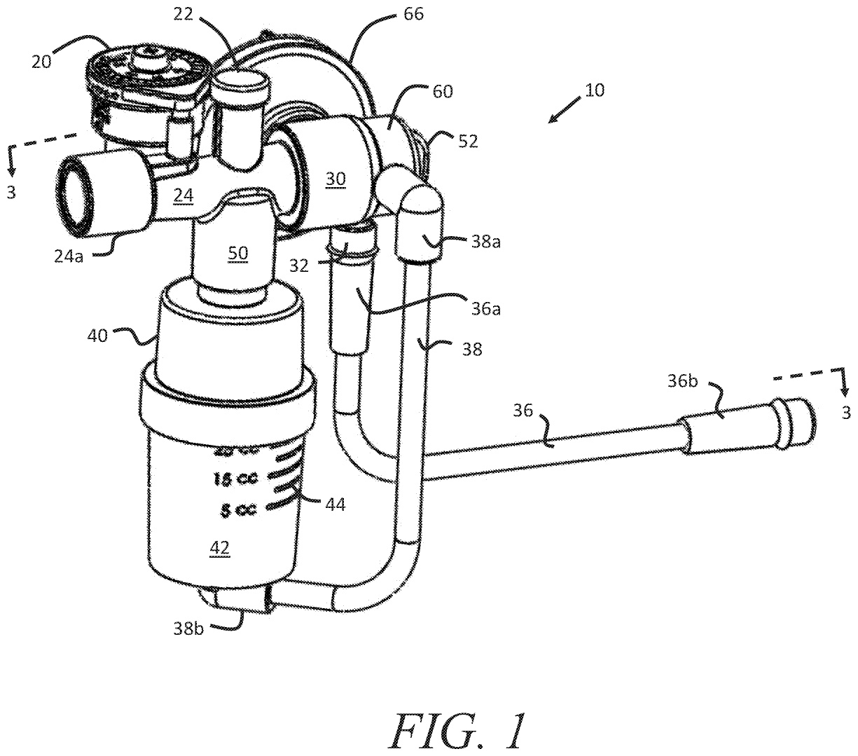

FIG. 1 depicts an embodiment of breathing assistance device (hereinafter “device”) 10. The device 10 includes manometer 20, pressure relief valve 22, and one or more filter devices 66. Patient breathing tube 24 is in open fluid communication at its leading end 24a with a face mask worn by a patient. Manometer 20 and pressure relief valve 22 are in open fluid communication with the patient breathing tube.

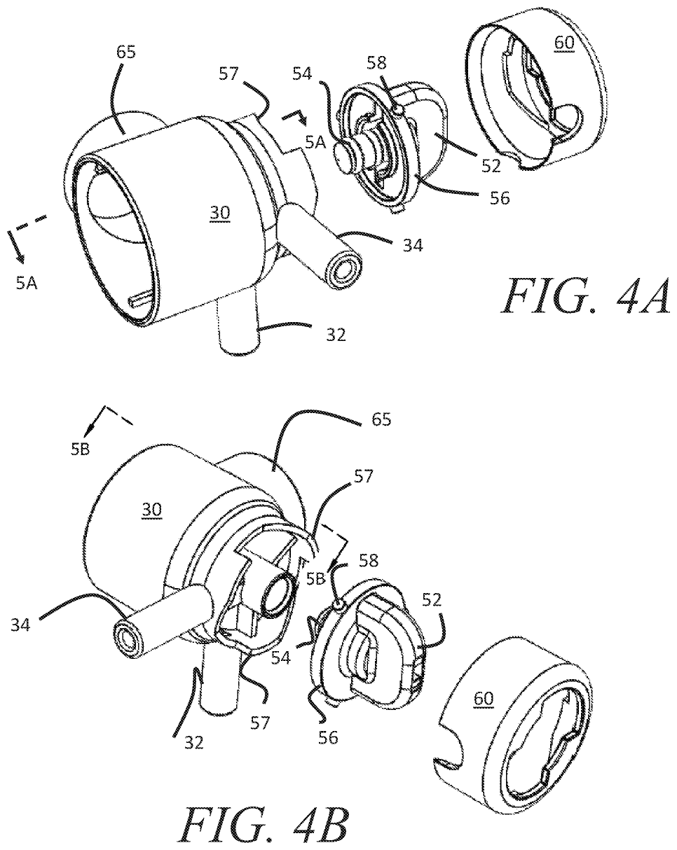

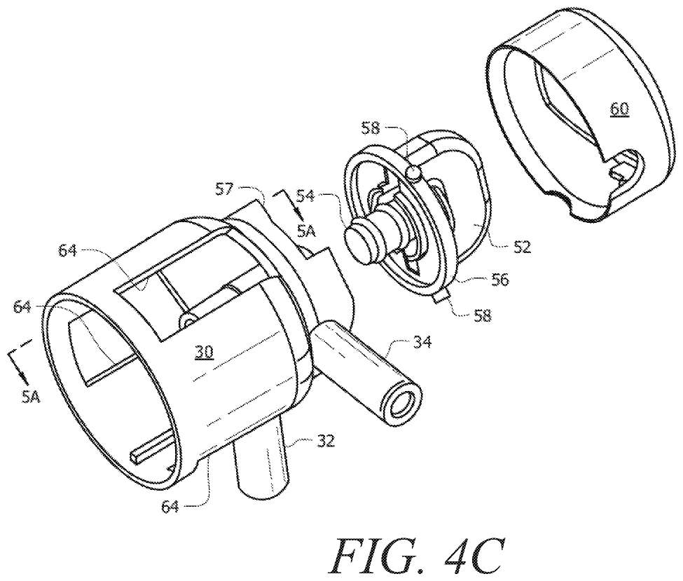

Valve body 30 is mounted to the trailing end of patient breathing tube 24. Ports 32 and 34 (see FIGS. 4A, 4B, and 4C) are preferably formed integrally with valve body 30 and are radially disposed relative to a longitudinal axis of valve body 30 to facilitate the attachment of conduits. Port 32 is vertically disposed and extends away from valve body 30. Port 34 is horizontally disposed with respect to port 32. Port 32 provides a mount for elongate, flexible conduit 36 that is connected at the enlarged trailing end 36b to a remote source of gaseous fluid under pressure, not depicted, and to port 32 at its enlarged leading end 36a. Port 34 provides a mount for elongate, flexible conduit 38 that is connected to nebulizer reservoir 42 at the enlarged trailing end 38b and to port 34 at its enlarged leading end 38a.

Nebulizer 40 includes a transparent or translucent reservoir 42 having graduation marks 44 imprinted thereon. A medical compound in liquid form and prescribed by a physician is charged into nebulizer reservoir 42 prior to the connection of leading end 24a of breathing tube 24 to a connecting port formed in a patient's face mask.

Nebulizer 40 atomizes the liquid fluid in nebulizer reservoir 42 into a mist or spray in a way that is well-known outside the context of breathing assistance devices, i.e., nebulizers are known for use in connection with spray bottles that dispense a wide variety of liquid fluids in spray or mist form.

Port 50 is preferably formed integrally with patient breathing tube 24 and depends vertically therefrom in parallel relation to port 32. The leading end of nebulizer 40 is mounted to port 50, such that the medical compound in compartment 42 is in spray or mist form when it flows through the patient's breathing tube 24.

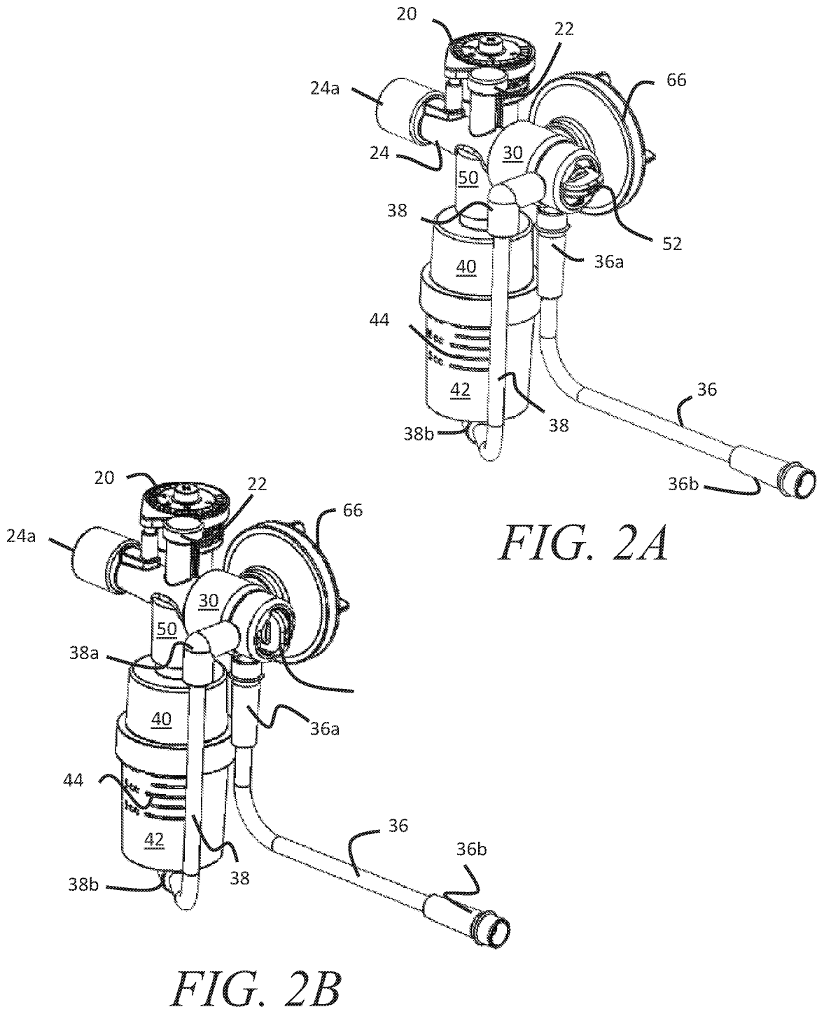

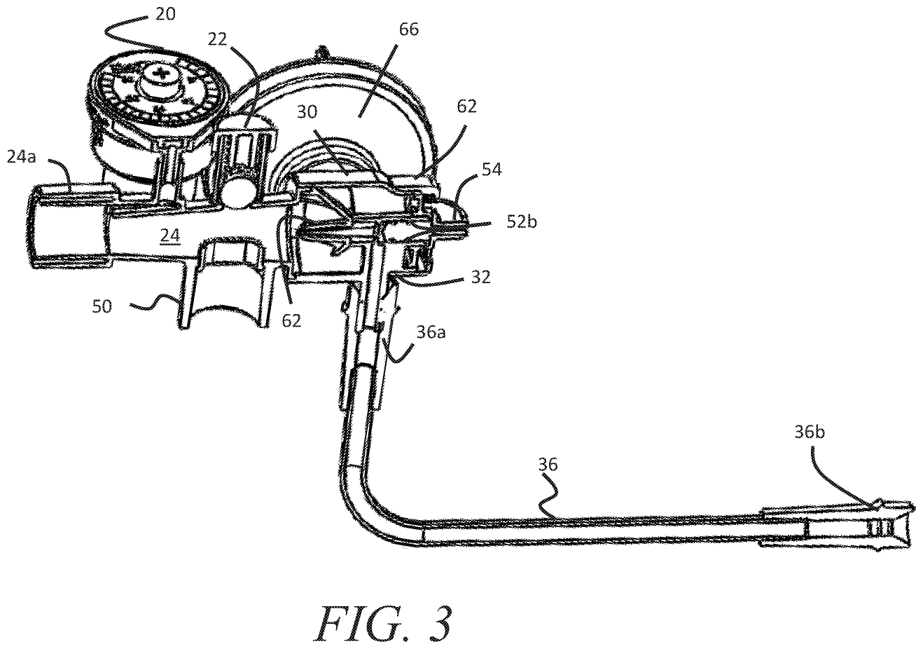

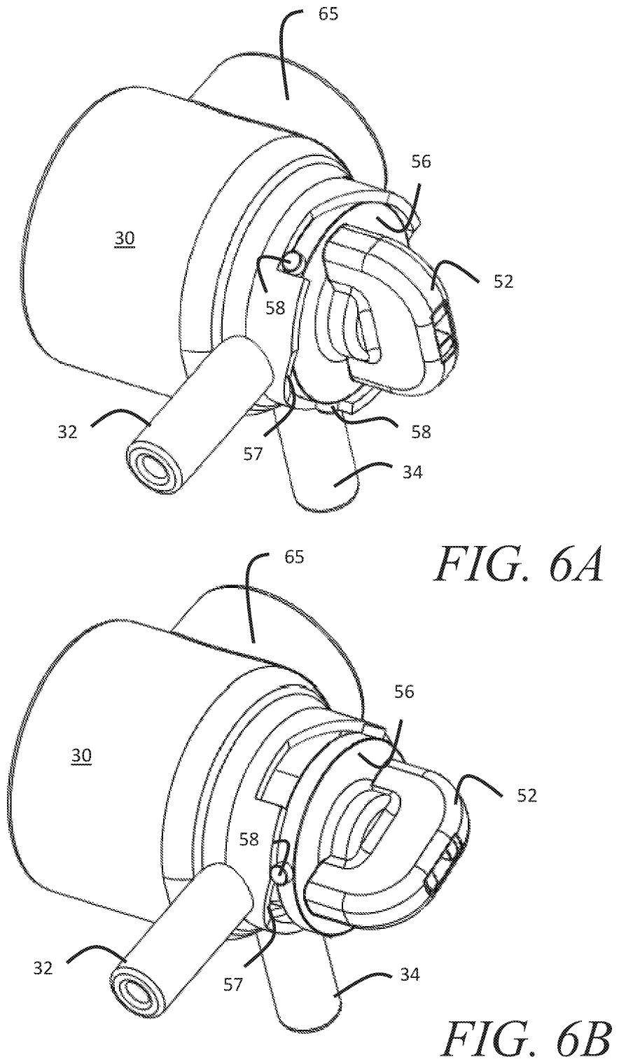

Valve actuator 52 is rotatably mounted relative to the longitudinal axis of symmetry of patient breathing tube 24 and valve 30. Depicted in FIGS. 2B and 6A, valve actuator 52 has a closed configuration that prevents the flow of gaseous fluid through tube 38 and an open configuration that enables the flow of gaseous fluid through tube 38. When valve actuator 52 is in the closed configuration, device 10 operates by delivering gaseous fluid under pressure to a patient. The gaseous fluid flows from the remote source of the gaseous fluid to the patient's face mask through conduit 36 and patient breathing tube 24. The closed configuration of valve actuator 52 prevents the flow of gaseous fluid through conduit 38, such that the liquid medication housed in nebulizer reservoir 42 is prevented from entering into the atomizing device of nebulizer 40.

When valve actuator 52 is in the open configuration, as depicted in FIGS. 1, 2A and 6B, gaseous fluid under pressure continues to flow from the remote source of gaseous fluid to the patient's face mask through patient breathing tube 24. The gaseous fluid also flows through tube 38 to nebulizer 40. This results in the fluid held within nebulizer reservoir 42 to flow through nebulizer 40 so that the liquid fluid is atomized into a mist or spray. The mist or spray is then introduced into the lumen of the patient's breathing tube 24 and entrained into the flow of gaseous fluid flowing through the patient's breathing tube 24 to the patient's face mask.

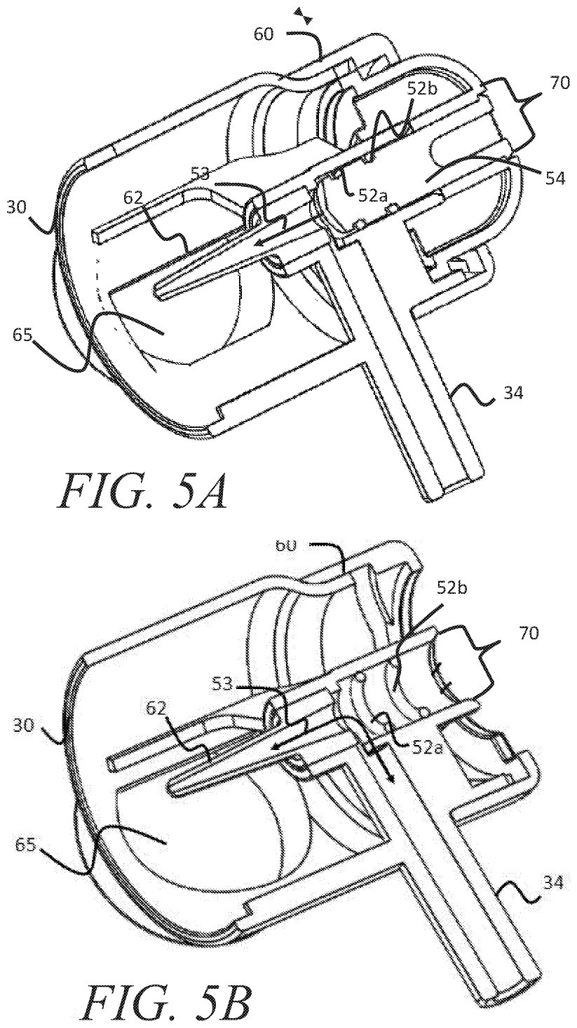

One or more O-rings 52a, 52b, depicted in FIGS. 5A and 5B, are mounted on shaft 54 and provide a sealing function when valve actuator 52 is in the closed configuration. More particularly, shaft 54 is axially displaced within channel 70 when valve actuator 52 is rotated ninety-degrees (90°). This is depicted by comparing the axial position of shaft 54 in FIG. 5A (shaft 54 extended, flow cut off) with the axial position of shaft 54 in FIG. 5B (shaft 54 retracted). Single-headed directional arrow 53 in FIG. 5A indicates the flow of gaseous fluid when shaft 54 is extended within channel 70. Double-headed directional arrow 55 in FIG. 5B indicates the flow of gaseous fluid when shaft 54 is retracted out of channel 70, such that valve actuator 52 is in the open configuration and gaseous fluid is flowing through both second tube 38 and first tube 36.

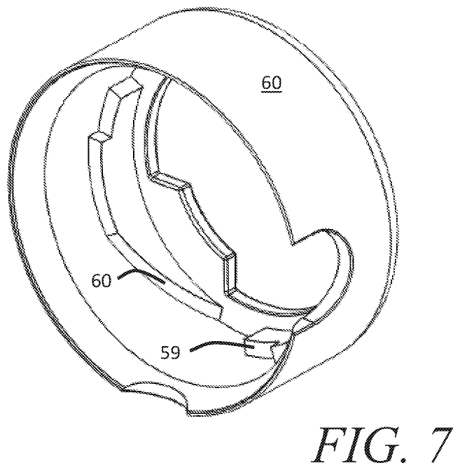

FIGS. 4B and 7 depict an embodiment of the invention having a plurality of circumferentially spaced-apart ramps 57 formed integrally with the trailing end of valve body 30. A plurality of circumferentially spaced apart pins 58 are formed integrally with disc 56. In an embodiment depicted in FIG. 4B, ramps 57 are depicted along with pins 58. Each pin 58 is formed integrally with disc 56 and extends perpendicularly away from a longitudinal axis of shaft 54. Each pin 58 translates up or down its respective ramp 57 depending on whether valve actuator is being opened or closed.

FIG. 7 depicts valve knob cover 60 having a plurality of ramps 59, formed therein that cooperate with ramps 57, such that when valve cover is secured to value body 30 each pin 58 is sandwiched between at least two ramps to constrain shaft 54 to be displaced axially along the longitudinal axis of symmetry as valve actuator 52 is rotated from the open configuration to the closed configuration or vice versa.

When valve actuator 52 is in its “nebulizer open” position, as depicted in FIGS. 1, 2A and 6B, the patient receives a combination of gaseous fluid from three sources: 1) orifice jet 62; 2) entrained room air through outlet port 65 formed within valve body 30; and 3) nebulizer 40, together with aerosolized medication. When valve actuator 52 is in its “nebulizer off” position, as depicted in FIGS. 2B and 6A, the patient receives a combination of gaseous fluid from two sources: 1) orifice jet 62; and 2) entrained room air through outlet port 65.

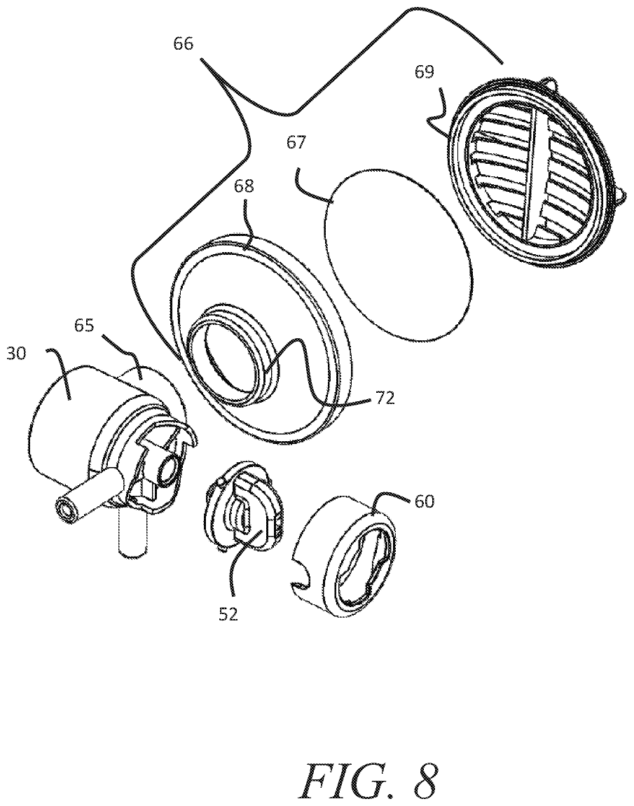

FIG. 8 depicts device 66 including housing 68 positioned adjacent to outlet port 65. In an embodiment, housing 68 may be a hollow geometric shape such as a frustoconical, triangular prism, cylindrical, or any number of other geometric shapes that one of ordinary skill in the art would appreciate to secure filter 67 therein when cover 69 is secured to housing 68. Housing 68 includes extension 72 disposed at a first end of housing 68 and has a complementary shape to that of outlet port 65, such that extension 72 may be secured to outlet port 65.

In an embodiment, extension 72 is secured to outlet port 65 by press-fitting within or over the top of an outer surface of outlet port 65 forming an airtight seal. Outlet port 65 or extension 72 may optionally include one or more O-rings disposed therein, further sealing the connection between outlet port 65 and extension 72. Extension 72 may be secured to outlet port 65 by any means known to a person of ordinary skill in the art. In addition, an embodiment includes extension 72 permanently secured or integrated with outlet port 65.

When the patient exhales, the gaseous fluid flows through outlet port 65 and through one or more filters 72, until the gaseous fluid is eventually discharged to the environment containing the patient and medical personnel.

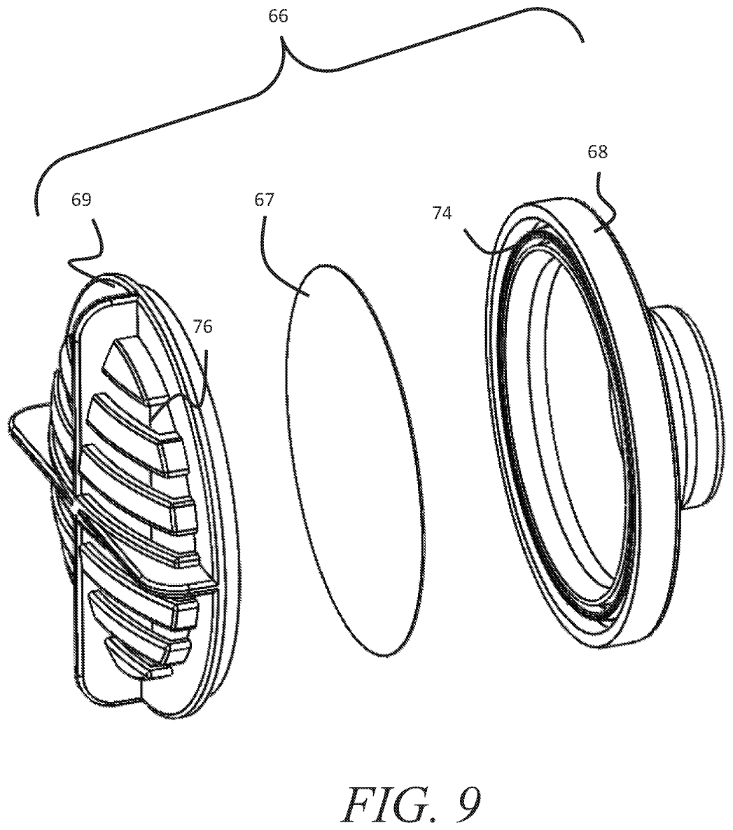

As shown in FIG. 9, the second end of housing 68 may optionally include O-ring 74 to provide an airtight seal when cover 69 is secured to the second end of housing 68. O-ring 74 ensures that the patient's exhaled breath is disposed through filters 67 and not leaked into the environment. In an embodiment, housing 68 may be secured to cover 69 using a clamp, adhesive, hook and loop fastener, thread and thread receipt, snap fitting, pressure fit, or any other method known in the art to secure cover 69 to housing 68. In an embodiment, housing 68 and cover 69 may be formed of a single piece, such as for example, through injection molding such that housing 68 and cover 69 are permanently secured or integrated with one another.

The filter results in the patient exhaling against a pressure slightly above atmospheric pressure due to the continuing incoming flow of gaseous fluids. In an embodiment, to prevent any further increase in pressure from affecting the patient's respiration, a hydrophobic filter 67 may be provided to permit water vapor naturally occurring during respiration from being absorbed into filter 67, thereby eliminating the chance for the pressure to build up when the patient naturally breathes.

Filters 67 may be provided, to filter a different particulate. In an embodiment, a single filter 67 may be used to filter bacteria, viruses, and/or unadministered medicine from the patient's breath. Filters 67 provide a protective barrier against the spread of pathogens and contaminates by preventing the patient's contaminated exhaled breath from being freely disseminated into an environment surrounding the patient. Filters 67 are shaped in substantially the same geometric configuration as the second end of the filter such that the majority of or all of the patient's exhaled breath must flow through filter 67.

In an embodiment, filter 67 may be those supplied by Hollingsworth & Vose Company™ In an embodiment, filter 67 may be composed of an electrostatically charged polypropylene and configured to filter out bacteria and/or viruses from the exhaled breath of the patient. In an embodiment, filters 67 are disposable and configured to be replaced after each use to provide a sanitary device for the next patient.

Subsequent to flowing through the one or more filters 67, the filtered gaseous fluid is exhausted through cover 69. The first end of cover 69 is complementary to the size and shape of the second end of the housing 68. The second end of cover 69 includes a protective portion 76 design to allow for the exhausting of the filtered gaseous fluid yet prevent objects from damaging filter 69 or being sucked into the device. In an embodiment, protective portion 76 may be in the form of slots, mesh, one or more apertures, or any other geometric shape, size, material, or configuration that one of ordinary skill in the art would appreciate facilitating the exhausting of filtered gaseous fluid and simultaneously preventing objects from contacting and damaging the one or more filters 67 housed within filter device 66. In an embodiment, cover 69 may be removably coupled to housing 68, such that the filters 67 may be easily swapped out and replaced depending on the specific medicine or medical ailment the device is being used to treat.

It will thus be seen that the objects set forth above, and those made apparent from the foregoing disclosure, are efficiently attained and since certain changes may be made in the above construction without departing from the scope of the invention, it is intended that all matters contained in the foregoing disclosure or shown in the accompanying drawings shall be interpreted as illustrative and not in a limiting sense.

It is also to be understood that the following claims are intended to cover all of the generic and specific features of the invention herein described, and all statements of the scope of the invention that, as a matter of language, might be the to fall therebetween.

What is claimed is:

1. A breathing assistance device, comprising:
a patient breathing tube having a leading end, a trailing end, and a patient breathing tube body extending therebetween, the leading end adapted to be in fluid communication with a facemask worn by a patient and the trailing end coupled to a valve body;
a nebulizer port disposed within the patient breathing tube body, the nebulizer port extending away from the patient breathing tube body from a first end to a second end;
a nebulizer coupled to the second end of the nebulizer port, the nebulizer including a reservoir secured to an engagement portion of the nebulizer, wherein the reservoir is adapted to contain a liquid;
a first port having a first end and a second end, the first end of the first port being coupled to the valve body;
a first tube having a trailing end connected in fluid communication to a gaseous fluid under pressure and a leading end coupled to a second end of the first port;
a second port having a first end and a second end, the first end being in valved communication with the valve body;
a second tube having a trailing end coupled with a second end of the second port and a leading end coupled to the reservoir, such that when the gaseous fluid flows through the second tube to the nebulizer, the fluid contained within the reservoir is atomized and introduced into the patient breathing tube, thereby permitting the patient to inhale both the gaseous fluid and the atomized fluid through the facemask; and
an outlet port disposed within at least a portion of the valve body, wherein the outlet port is secured to a filter device, such that when the patient exhales, the contaminated gaseous fluid is exhausted to an environment surrounding the patient through the filter device.

2. The breathing assistance device of claim 1, further comprising:
a valve actuator having a closed configuration and an open configuration;
the closed configuration permits the gaseous fluid to flow through the first tube and restricts the flow of the gaseous fluid through the second tube; and
the open configuration permits the gaseous fluid to flow through the first tube and the second tube, such that at least a portion of the gaseous fluid flows through the nebulizer.

3. The breathing assistance device of claim 2, wherein the valve actuator further includes a shaft having a pair of longitudinally spaced O-rings at least partially disposed within an outer surface of the shaft.

4. The breathing assistance device of claim 3, wherein the shaft is axially displaced when the valve actuator is rotated from a first position to a second position.

5. The breathing assistance device of claim 4, further comprising:
a plurality of ramps formed integrally with the trailing end of the valve body;
the valve actuator having a disc formed integrally therewith, the disc being disposed transverse to a longitudinal axis of the valve body;
a plurality of pins formed integrally with the disc and projecting radially therefrom;
a valve body cover having a plurality of ramps formed therein that match in quantity the number of ramps formed in the trailing end of the valve body;
the pins being sandwiched between the ramps formed in the valve body and the ramps formed in the valve body cover to constrain the shaft to displace axially along the longitudinal axis as the valve actuator is rotated;
the pins sliding up their respective ramps when the valve actuator is rotated from the first position to the second position and the pins sliding down their respective ramps when the valve actuator is rotated from the second position to the first position.

6. The breathing assistance device of claim 1, further comprising an orifice jet disposed concentrically within a lumen of the patient breathing tube and in fluid communication with the gaseous fluid when the valve actuator is in either the open position or the closed position.

7. The breathing assistance device of claim 1, wherein the first and the second ports extend radially from the valve body in a circumferentially spaced relation to one another.

8. The breathing assistance device of claim 1, wherein the liquid is a medicinal compound in liquid form.

9. The breathing assistance device of claim 1, wherein at least a first filter and a second filter are configured to reside within the filter device, each of the first filter and the second filter.

10. The breathing assistance device of claim 1, wherein the filter device further comprises:
a housing having a first end and a second end, at least a portion of the first end of the housing includes an extension extending away from the first end of the housing and secured to the outlet port of the valve body, thereby creating an airtight seal between the outlet port and the housing;
a cover having a first side and a second side, the first side configured to be removably secured to the second end of the housing; and
a filter residing between the second end of the housing and the first side of the cover, such that the gaseous fluid passes through the filter before exhausted through the second side of the cover.

11. A device to assist a patient in breathing, the device comprising:
a patient breathing tube having a leading end, a trailing end, and a patient breathing tube body extending therebetween, the leading end coupled with a facemask worn by a patient and the trailing end coupled with a valve body;
a nebulizer port disposed within the patient breathing tube body, the nebulizer port extending away from the patient breathing tube body from a first end to a second end;
a nebulizer coupled to the second end of the nebulizer port, the nebulizer including a reservoir coupled to an engagement portion of the nebulizer, wherein the reservoir is adapted to contain a medicinal compound in liquid form;
a first port having a first end and a second end, the first end of the first port being coupled to the valve body;
a first tube having a trailing end connected in fluid communication to a gaseous fluid under pressure and a leading end coupled to a second end of the first port;
a second port having a first end and a second end, the first end being in valved communication with the valve body;
a second tube having a trailing end coupled with a second end of the second port and a leading end coupled to the reservoir, such that when the gaseous fluid flows through the second tube to the nebulizer, the medicinal compound contained within the reservoir is atomized and introduced into the patient breathing tube, thereby permitting the patient to inhale both the gaseous fluid and the atomized medicinal compound through the facemask; and
an outlet port disposed within at least a portion of the valve body, wherein the outlet port is secured to a filter device, the filter device comprising:
a housing having a first end and a second end, at least a portion of the first end of the housing includes an extension extending away from the first end of the housing and secured to the outlet port of the valve body thereby creating an airtight seal between the outlet port and the housing;
a cover having a first side and a second side, the first side configured to be removably secured to the second end of the housing; and
a filter residing between the second end of the housing and the first side of the cover, such that the gaseous fluid passes through the filter before exhausted through the cover;
wherein exhalation of the contaminated gaseous fluid by the patient disposes the contaminated gaseous fluid through the filter device prior to being exhausted through the cover.

12. The device of claim 11, further comprising:
a valve actuator having a closed configuration and an open configuration;
the closed configuration permits the gaseous fluid to flow through the first tube and restricts the flow of the gaseous fluid through the second tube; and
the open configuration permits the gaseous fluid to flow through the first tube and the second tube, such that at least a portion of the gaseous fluid flows through the nebulizer prior to being inhaled by the patient.

13. The breathing assistance device of claim 12, wherein the valve actuator further includes a shaft configured to be axially displaced when the valve actuator is rotated from a first position to a second position, the shaft including a pair of longitudinally spaced O-rings at least partially disposed within an outer surface of the shaft.

14. The breathing assistance device of claim 13, further comprising:
a plurality of ramps formed integrally with the trailing end of the valve body;
the valve actuator having a disc formed integrally therewith, the disc being disposed transverse to a longitudinal axis of the valve body;
a plurality of pins formed integrally with the disc and projecting radially therefrom;
a valve body cover having a plurality of ramps formed therein that match in quantity the number of ramps formed in the trailing end of the valve body;
the pins being sandwiched between the ramps formed in the valve body and the ramps formed in the valve body cover to constrain the shaft to displace axially along the longitudinal axis as the valve actuator is rotated;
the pins sliding up their respective ramps when the valve actuator is rotated from the first position to the second position and the pins sliding down their respective ramps when the valve actuator is rotated from the second position to the first position.

15. The device of claim 11, further comprising an orifice jet disposed concentrically within a lumen of the patient breathing tube and in fluid communication with the gaseous fluid when the valve actuator is in either the open position or the closed position.

16. A device to assist a patient in breathing, the device comprising:
a patient breathing tube having a leading end, a trailing end, and a patient breathing tube body extending therebetween, the leading end coupled to a facemask worn by a patient and the trailing end coupled with a valve body;
a nebulizer port disposed within the patient breathing tube body, the nebulizer port extending away from the patient breathing tube body from a first end to a second end;
a nebulizer coupled to the second end of the nebulizer port, the nebulizer including a reservoir coupled to an engagement portion of the nebulizer, wherein the reservoir is adapted to contain a medicinal compound in liquid form;
a first port having a first end and a second end, the first end of the first port being coupled to the valve body;
a first tube having a trailing end connected in fluid communication to a gaseous fluid under pressure and a leading end coupled to a second end of the first port;
a second port having a first end and a second end, the first end being in valved communication with the valve body;
a second tube having a trailing end coupled with a second end of the second port and a leading end coupled to the reservoir, such that when the gaseous fluid flows through the second tube to the nebulizer, the medicinal compound contained within the reservoir is atomized and introduced into the patient breathing tube, thereby permitting the patient to inhale both the gaseous fluid and the atomized medicinal compound through the facemask; and
an outlet port disposed within at least a portion of the valve body, wherein the outlet port is secured to a filter device comprising:
a housing having a first end and a second end, at least a portion of the first end of the housing includes an extension extending away from the first end of the housing and secured to the outlet port of the valve body, thereby creating an airtight seal between the outlet port and the housing;
a cover having a first side and a second side, the first side configured to be removably secured to the second end of the housing; and
a first filter and a second filter residing between the second end of the housing and the first side of the cover, such that the gaseous fluid passes through each of the first and the second filters before the gaseous fluid is exhausted through the cover;
wherein exhalation of the gaseous fluid by the patient disposes the gaseous fluid through the filter device prior to being exhausted through the cover, such that the first and the second filters are configured to filter harmful contaminates from the gaseous fluid being exhausted.

17. The breathing assistance device of claim 16, further comprising:
a valve actuator having a closed configuration and an open configuration;
the closed configuration permits the gaseous fluid under pressure to flow through the first tube and restricts the flow of the gaseous fluid under pressure through the second tube; and
the open configuration permits the remote source of gaseous fluid to flow through the first tube and the second tube.

18. The breathing assistance device of claim 17, wherein the valve actuator further includes a shaft configured to be axially displaced when the valve actuator is rotated from a first position to a second position, the shaft including a pair of longitudinally spaced O-rings at least partially disposed within the outer surface of the shaft.

19. The breathing assistance device of claim 18, further comprising:
a plurality of ramps formed integrally with the trailing end of the valve body;
the valve actuator having a disc formed integrally therewith, the disc being disposed transverse to a longitudinal axis of the valve body;
a plurality of pins formed integrally with the disc and projecting radially therefrom;
a valve body cover having a plurality of ramps formed therein that match in quantity the number of ramps formed in the trailing end of the valve body;
the pins being sandwiched between the ramps formed in the valve body and the ramps formed in the valve body cover to constrain the shaft to displace axially along the longitudinal axis as the valve actuator is rotated;
the pins sliding up their respective ramps when the valve actuator is rotated from the first position to the second position and the pins sliding down their respective ramps when the valve actuator is rotated from the second position to the first position.

20. The breathing assistance device of claim 16, further comprising an orifice jet disposed concentrically within a lumen of the patient breathing tube and in fluid communication with the gaseous fluid when the valve actuator is in either the open position or the closed position.
